(19) United States
(12) Patent Application Publication
(10) Pub. No.: US 2007/0021359 A1
(43) Pub. Date: Jan. 25, 2007

(54) Addition salts of azithromycin and citric acid and process for preparing them

(76) Inventors: Antonio Cosme Gomez, Torres De Alameda (ES); Francisco Eugenio Palomo Nicolau, Alacala De Henares (ES)

Correspondence Address: WOLF BLOCK SCHORR AND SOLIS-COHEN LLP, 250 PARK AVENUE, NEW YORK, NY, 10177, US

(21) Appl. No.: 10/558,801

(22) PCT Filed: May 26, 2004
(86) PCT No.: PCT/IB04/01728

§ 371 Date: Nov. 29, 2005

(30) Foreign Application Priority Data
May 29, 2003 (ES) P-200301340

(51) Int. Cl. A61K 31/7052; C07H 17/08

(52) U.S. Cl. 514/28; 536/7.4

(57) ABSTRACT

Said addition salts have a molar ratio between azithromycin and citric acid such as to provide a pH comprised between 4.0 and 8.0 in a 10% aqueous solution. The process for preparing said salts comprises: a) dissolving azithromycin in a solvent or mixture of solvents; b) adding citric acid; and c) isolating the product obtained by crystallisation. The addition salts of azithromycin and citric acid are stable and soluble in aqueous medium, being useful antibacterial and antiprotozoan agents.

### FIELD OF THE INVENTION

This invention relates to new addition salts of azithromycin and citric acid, their preparation, their use in pharmaceutical compositions and the aqueous or water-alcohol solutions containing them, as well.

### BACKGROUND OF THE INVENTION

Azithromycin or 9-deoxo-9a-aza-9a-methyl-9a-homoerythromycin A:

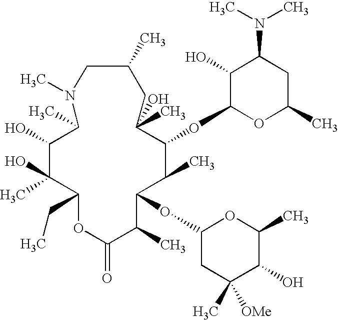

is a broad-spectrum antibacterial agent which was described and patented by Sour Pliva in Yugoslavian patent application YU 000592 of Jun. 3, 1981, priority claimed in the equivalent U.S. Pat. No. 4,517,359.

On the other hand, European patent EP 298650 describes azithromycin monohydrate and azithromycin dihydrate. Chinese patents CN 1123279A, CN 1157824A and CN 1205338A, describe methods for preparing azithromycin salts with organic and inorganic acids. The publication J. Chem. Research (M), 1988, 1239-1261; J. Chem. Research (S), 1988, 152-153 describe azithromycin dihydrochloride, dihydroiodide, diacetate, diaspartate, diglucoheptonate and dilactobionate. Patent application WO 00/32203 discloses azithromycin ethanolate and European patent application EP 984020 discloses an isopropanol caltrate of azithromycin monohydrate. Patent application WO 02/094843 discloses various crystalline forms of azithromycin, characterised by the carbon 13 nuclear magnetic resonance spectrum (¹³C-NMR) and the X-ray diffraction spectrum.

It is known that azithromycin is not stable in an aqueous acid medium, and furthermore base azithromycin is very insoluble in water.

There is therefore a need for providing new acid addition salts of azithromycin that are soluble in aqueous medium while at the same time having suitable stability properties in solid phase and in solution.

### BRIEF DESCRIPTION OF THE INVENTION

The object of this invention is to provide new addition salts of azithromycin and citric acid soluble in aqueous medium while at the same time having suitable stability properties in solid phase and in solution.

A further object of this invention is to provide a process that is useful for preparing such salts and their use for therapeutic purposes.

### BRIEF DESCRIPTION OF THE FIGURES

FIG. 1 shows the X-ray diffraction spectrum of azithromycin hydrogen citrate.

FIG. 2 shows the carbon 13 nuclear magnetic resonance spectrum (¹³C-NMR) of azithromycin hydrogen citrate in solid state.

FIG. 3 shows the IR spectrum of azithromycin hydrogen citrate, recorded on KBr tablet.

FIG. 4 shows the X-ray diffraction spectrum of azithromycin hydrogen citrate.

FIG. 5 shows the carbon 13 nuclear magnetic resonance spectrum (¹³C-NMR) of azithromycin hydrogen citrate in solid state.

FIG. 6 shows the IR spectrum of azithromycin hydrogen citrate, recorded on KBr tablet.

FIG. 7 shows the X-ray diffraction spectrum of azithromycin hydrogen citrate.

FIG. 8 shows the carbon 13 nuclear magnetic resonance spectrum (¹³C-NMR) of azithromycin hydrogen citrate in solid state.

FIG. 9 shows the IR spectrum of azithromycin hydrogen citrate, recorded on KBr tablet.

FIG. 10 shows the X-ray diffraction spectrum of azithromycin citrate.

FIG. 11 shows the carbon 13 nuclear magnetic resonance spectrum (¹³C-NMR) of azithromycin citrate in solid state.

FIG. 12 shows the IR spectrum of azithromycin citrate, recorded on KBr tablet.

FIG. 13 shows the X-ray diffraction spectrum of azithromycin citrate.

FIG. 14 shows the carbon 13 nuclear magnetic resonance spectrum (¹³C-NMR) of azithromycin citrate in solid state.

FIG. 15 shows the IR spectrum of azithromycin citrate, recorded on KBr tablet.

### DETAILED DESCRIPTION OF THE INVENTION

Surprisingly, the authors of this invention have found new addition salts of azithromycin and citric acid which show good solubility in aqueous medium and good stability properties in solid phase and in solution.

In a first aspect, this invention relates to a new addition salt of azithromycin and citric acid, the molar ratio between the azithromycin and the citric acid being such as to provide a pH between 4.0 and 8.0 in a 10% aqueous solution.

In one embodiment of the invention, said salt is azithromycin hydrogen citrate, which is characterised in that it has a molar ratio of azithromycin and citric acid such as to provide a pH between 4.0 and 6.0 in 10% aqueous solution.

For the purposes of the present invention and except where expressly stated otherwise, the percentage of the addition salt of azithromycin and citric acid in aqueous solution is expressed in weight/weight or weight/volume.

Preferably, the azithromycin hydrogen citrate salt contains up to 8% water, more preferably up to 6%, under relative humidity conditions of 40%.

More preferably still, said azithromycin hydrogen citrate further contains up to 3% of residual solvent.

Advantageously, said azithromycin hydrogen citrate is characterised in that it has a molar ratio of azithromycin and citric acid close to the stoichiometric ratio that provides a pH of 5 in a 10% aqueous solution.

In a second embodiment of the invention, said salt is azithromycin citrate, which is characterised by having a molar ratio of azithromycin and citric acid such as to provide a pH between 6.0 and 8.0 in 10% aqueous solution.

Preferably, the azithromycin citrate salt contains up to 8% water, and more preferably still up to 6%, under relative humidity conditions of 40%.

More preferably still, the azithromycin citrate further contains up to 3% of residual solvent.

Advantageously, said azithromycin citrate has a molar ratio of azithromycin and citric acid of 3:2.

Also advantageously, the azithromycin citrate, in accordance with one preferable embodiment of the present invention, is in amorphous form.

The azithromycin citrate in accordance with one embodiment of the invention is characterised in that it has a chemical combination of one molecule of azithromycin per ⅔ of a molecule of citric acid (chemically, 3 moles of azithromycin and 2 moles of citric acid), resulting in a neutral salt in which the basic groups of azithromycin (two equivalents) form a salt with the acid groups of the citric acid (3 equivalents).

The azithromycin citrate of the invention provides aqueous solutions up to 65% (w/w) at ambient temperature, with a pH between 6.8 and 7.5.

A second aspect of the invention is to provide a process for preparing an addition salt of azithromycin and citric acid, in accordance with the first aspect of this invention. Such process comprises: a) dissolving azithromycin in a solvent or mixture of solvents, b) adding citric acid; and c) isolating the product obtained.

Citric acid or 2-Hidroxy-1,2,3-propanotricarboxylic acid is a carboxylic acid that has three COOH groups in its molecule.

Azithromycin has two nitrogen groups of basic nature in its molecule and for the process of the invention can be used either in monohydrate or dihydrate form of azithromycin.

In one embodiment of the process of the invention, step (a) is carried out by dissolving azithromycin in monohydrated form.

In another embodiment, step (a) is carried out by dissolving azithromycin in dihydrated form.

For the purposes of this invention, unless expressly stated otherwise, dissolving azithromycin in a solvent or mixture of solvents should be understood to mean any degree of dissolution, with total dissolution of the product at the start of the process being unnecessary.

The addition salt of azithromycin and citric acid can be prepared in practically any kind of solvent, although it is more difficult its preparation in solvents in which both molecules are insoluble (for example, in toluene or heptane). The following can be used as solvents: water; linear or branched C₁-C₆ aliphatic alcohols, such as methanol, ethanol, n-propanol, isopropanol, n-butanol, etc.; cyclic aliphatic alcohols, such as cyclohexanol; diols, such as ethylene glycol, 1,2-propylene glycol, 1,3-propanodiol, 1,4-butanodiol, etc.; linear or branched C₁-C₆ aliphatic ketones, such as acetone, methyl ethyl ketone, methyl isobutyl ketone, etc.; cyclic aliphatic ketones, such as cyclohexanone; short-chain aliphatic esters, such as methyl or ethyl acetate; short-chain aliphatic ethers, such as ethylic ether, isopropylic ether, etc.; cyclic aliphatic ethers, such as tetrahydrofuran and dioxane, or mixtures thereof.

In one embodiment of the process of the invention, the azithromycin hydrogen citrate salt is prepared by isolating the salt by means of crystallisation in step (c).

The following aspects, independently or together, are preferred in the preceding embodiment: the azithromycin is selected from the azithromycin monohydrate or dihydrate; the molar proportions of azithromycin and citric acid are close to the stoichiometric; the solvents are selected from alcohols, ketones, esters or ethers or mixtures thereof, preferably ethanol, acetone, methyl acetate or tetrahydrofuran or mixtures thereof; the crystallisation temperature is between 25° C. and the solvent's reflux temperature; and the mixture is cooled to a temperature between 0° C. and 25° C. before separating the crystals.

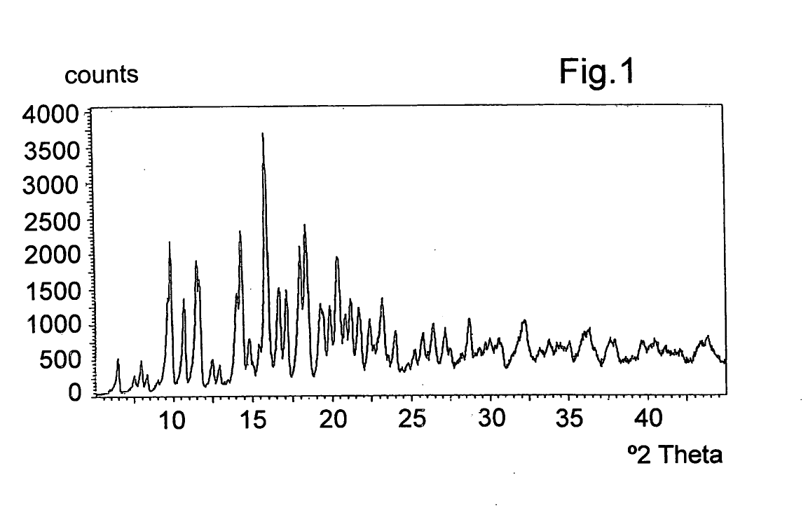

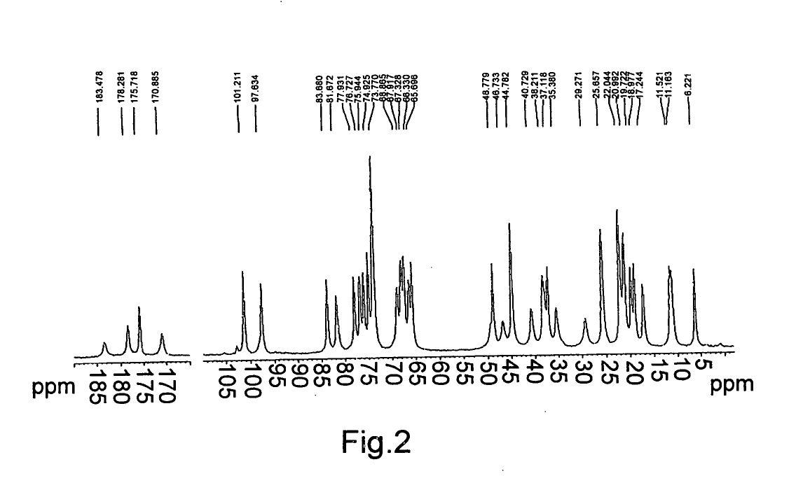

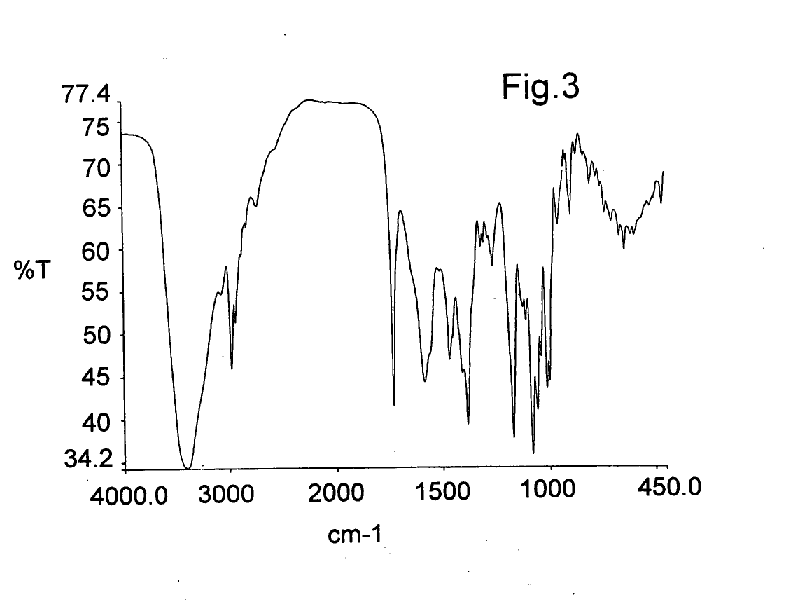

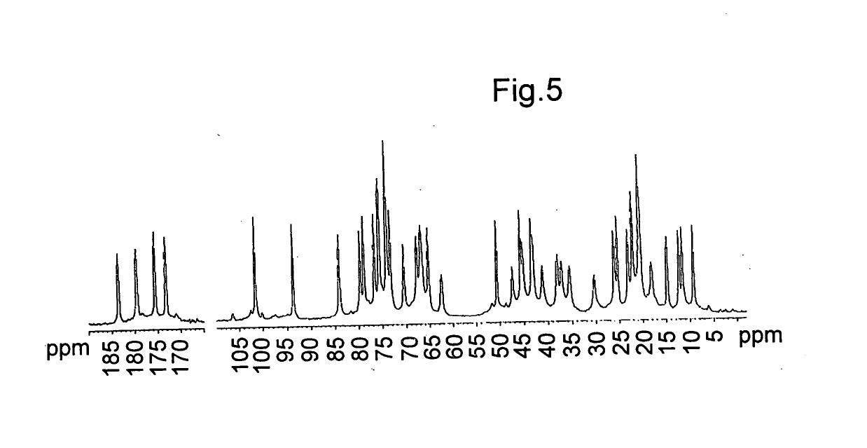

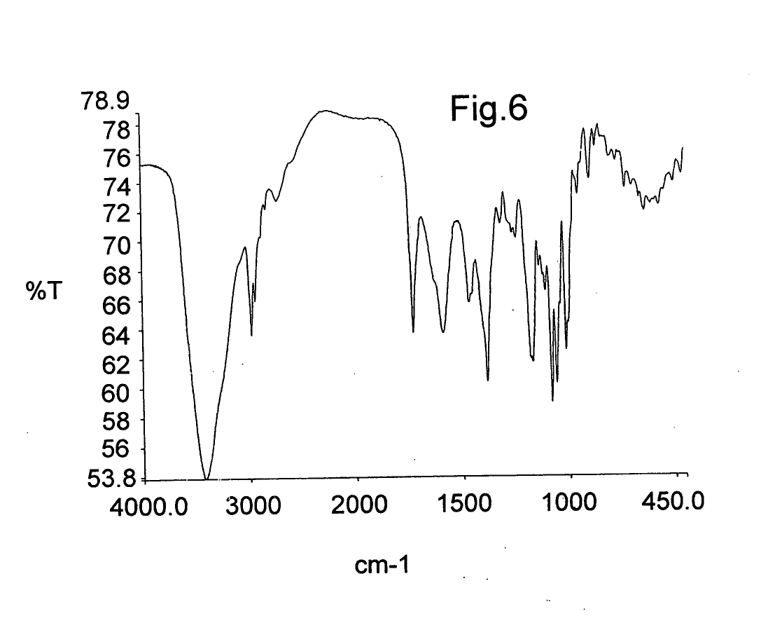

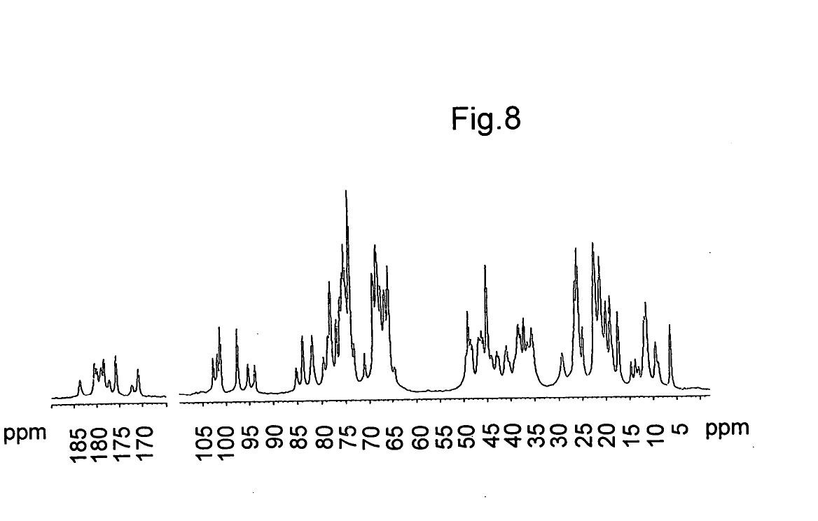

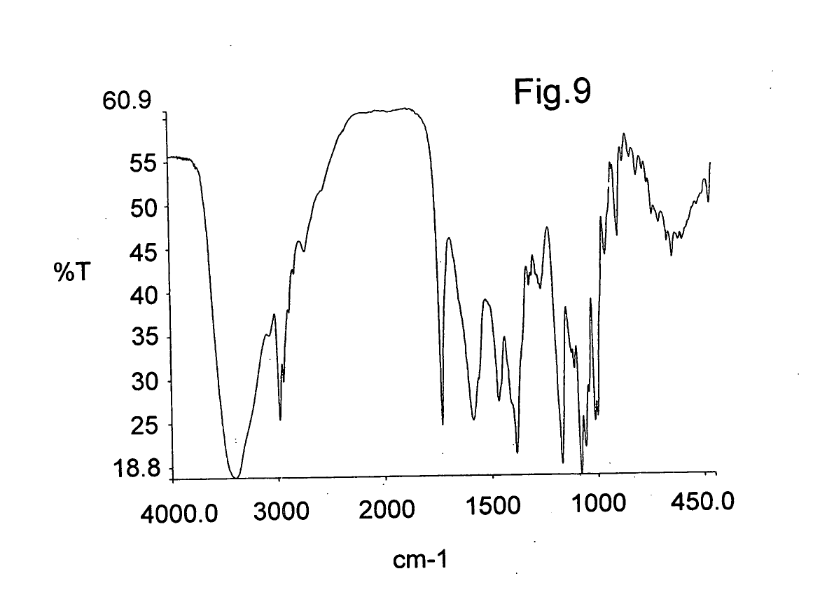

The X-ray diffraction, carbon 13 nuclear magnetic resonance (¹³C-NMR) in solid state and IR spectra serve to identify the azithromycin hydrogen citrate in accordance with the first aspect of the invention. See FIGS. 1 to 9.

In another embodiment of the process of the invention, the azithromycin citrate is prepared by adding an amount of citric acid in step (b) such that the molar ratio between the azithromycin and the citric acid is 3:2.

Advantageously, when the azithromycin citrate is prepared, the salt is isolated in step (c) by eliminating the solvent.

The following aspects, independently or together, are preferred in the preceding embodiment: the azithromycin is selected from the azitromycin monohydrate or dihydrate; the solvents are selected from water, alcohols, ketones, esters or ethers, or mixtures thereof, preferably water, ethanol, acetone, methyl acetate or tetrahydrofuran, or mixtures thereof.

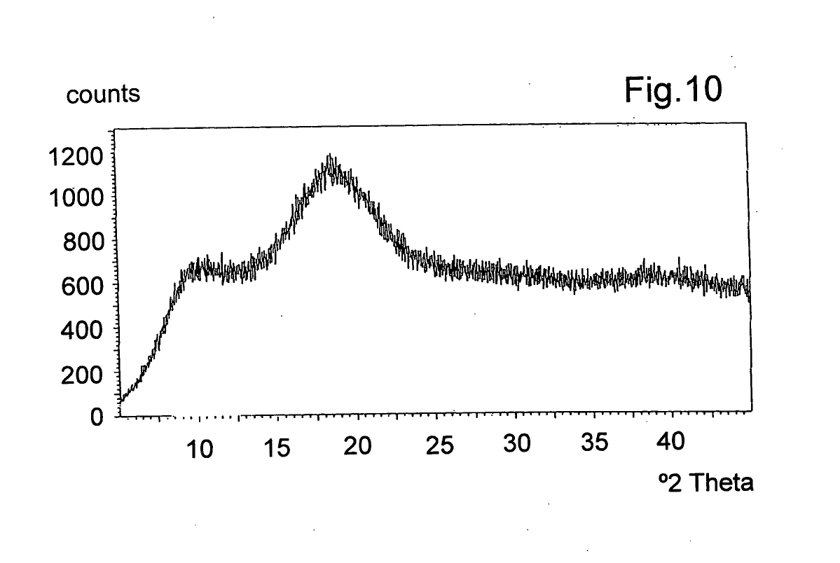

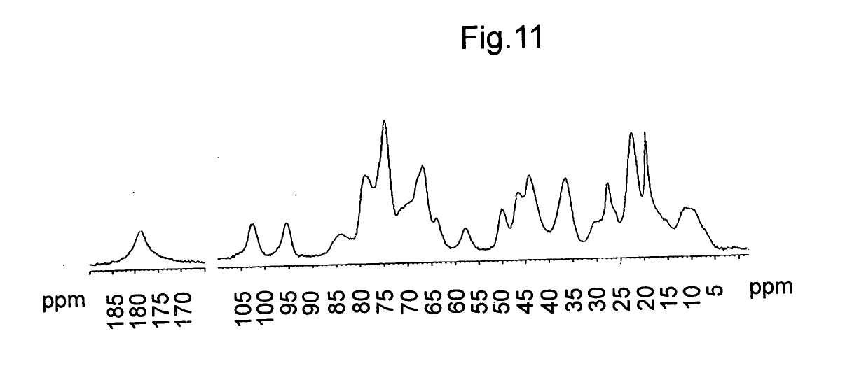

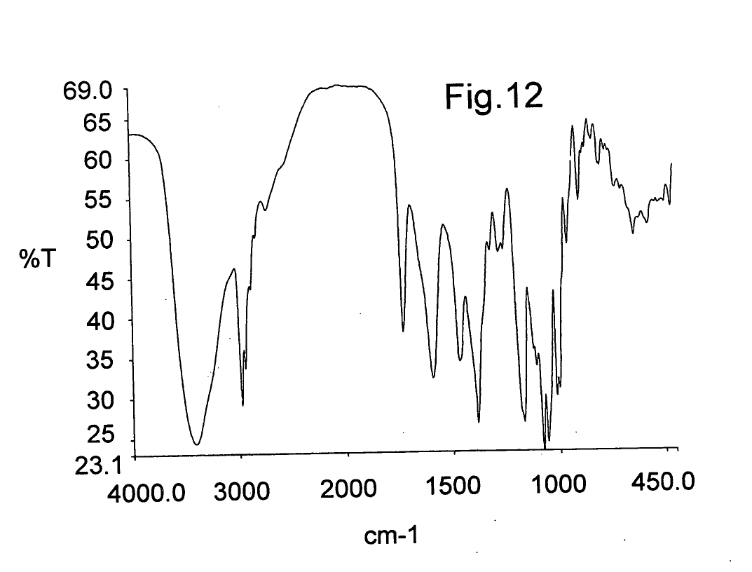

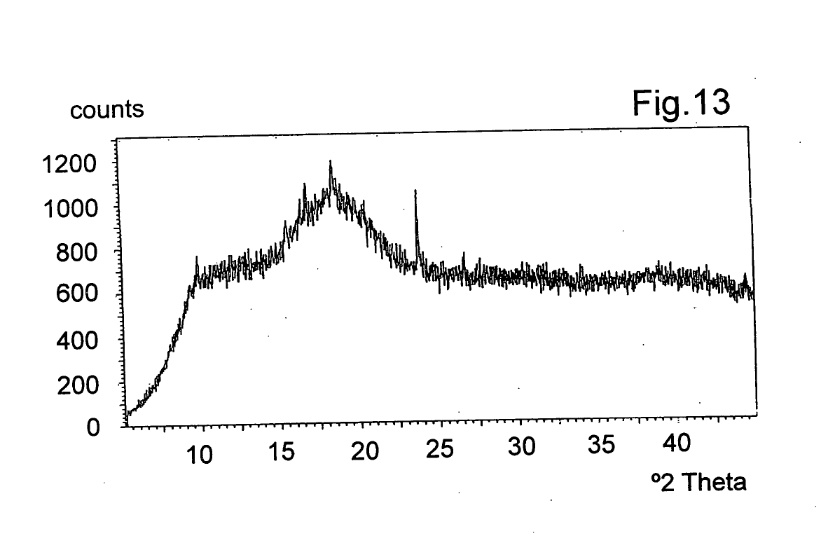

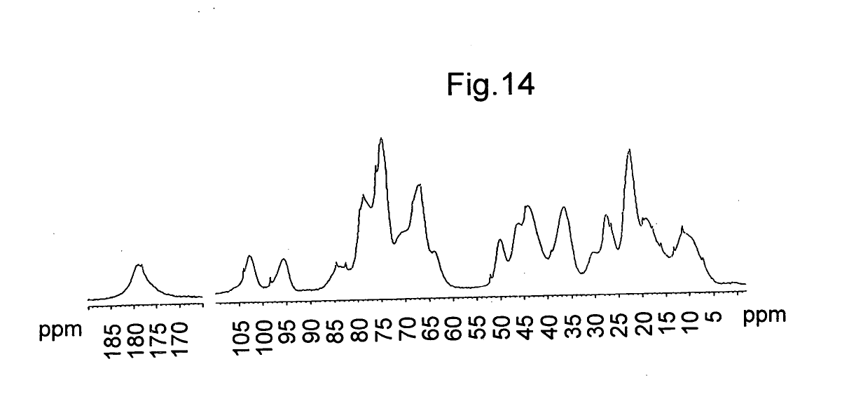

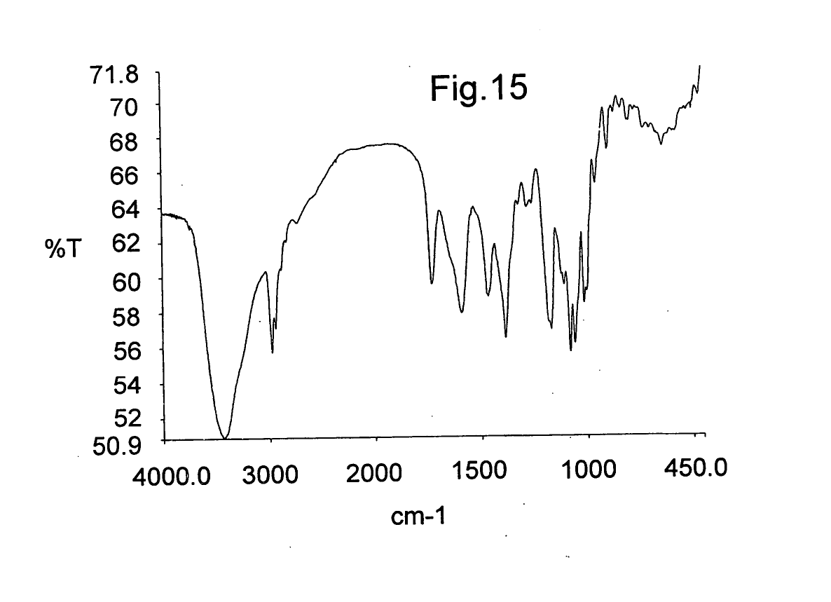

The X-ray diffraction, carbon 13 nuclear magnetic resonance (¹³C-NMR) in solid state and IR spectra serve to identify the azithromycin citrate produced in accordance with the invention. See FIGS. 10 to 15.

The new aqueous-medium-soluble addition salts of azithromycin and citric acid of the invention that have suitable stability characteristics in solid phase and in solution are useful as antibacterial and antiprotozoans. They can be administered orally, parenterally, topically or rectally in the treatment or prevention of infections caused by bacteria or protozoa.

The new addition salts of azithromycin and citric acid of the invention are particularly useful in the preparation of aqueous or water-alcohol solutions of azitromycin containing up to 65% of the salt, providing a pH between 4 and 8, stable and not suffering from chemical degradation of azithromycin.

For a better understanding of all that has been described there follow some examples which show, schematically and solely by way of non-restrictive example, some embodiments of the invention.

### EXAMPLES

### Example 1

### Preparation of Azithromycin Hydrogen Citrate

20 g of azithromycin are added to 100 ml of acetone (water content according to Karl-Fisher of 1 to 5%), the mixture is stirred at ambient temperature until dissolved. 5.35 g of citric acid are added and the mixture is heated at reflux. It is then cooled to 0-5° C., filtered, washed with acetone and dried under vacuum at 40° C. to yield 22.4 g of azithromycin hydrogen citrate (water content according to Karl-Fisher of 1.2% and acetone content less than 0.5%). The azithromycin content determined by HPLC is 80% and the citric acid content by electrometric titration is 20%, corresponding to the stoichiometric ratio of the azithromycin hydrogen citrate. The salt can contain up to 8% water depending on the drying method (by vacuum, fluidised bed, static), but is preferably 6%, under relative humidity conditions of 40%. FIGS. 1, 2 and 3 show the X-ray diffraction spectrum, the carbon 13 nuclear magnetic resonance spectrum (¹³C-NMR) in solid state and the IR spectrum, recorded on KBr tablet, respectively.

### Example 2

### Preparation of Azithromycin Hydrogen Citrate

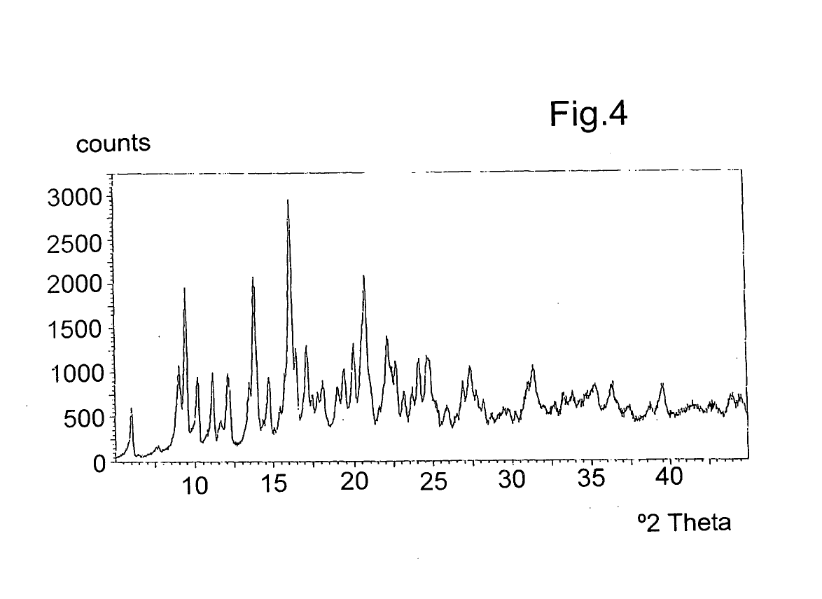

20 g of azithromycin dihydrate and 3.5 g of citric acid monohydrate are added to 50 ml of methyl acetate. This is heated at reflux, cooled to ambient temperature, filtered, washed with methyl acetate and dried under vacuum at 40° C. FIGS. 4, 5, and 6 show the X-ray diffraction spectrum, the carbon 13 nuclear magnetic resonance spectrum (¹³C-NMR) in solid state and the IR spectrum, recorded on KBr tablet, respectively.

### Example 3

### Preparation of Azithromycin Hydrogen Citrate

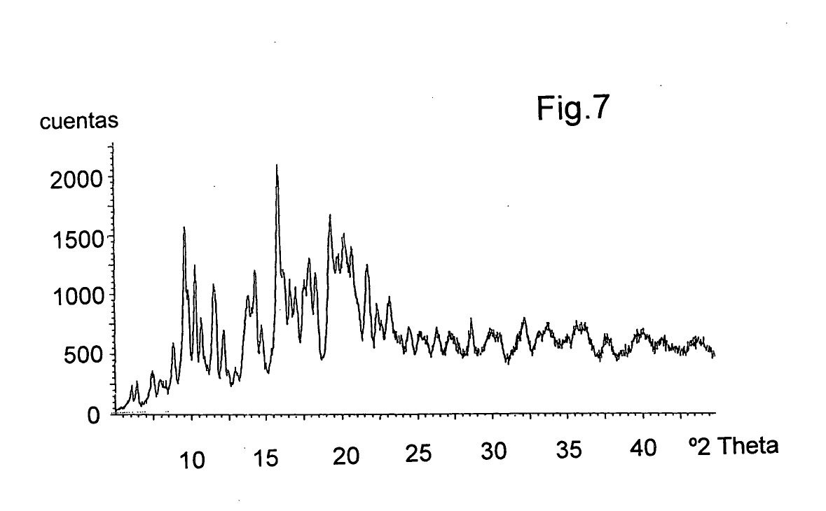

Following the procedure set out in example 2 and replacing the methyl acetate by tetrahydrofuran, azithromycin hydrogen citrate is obtained. FIGS. 7, 8 and 9 show the X-ray diffraction spectrum, the carbon 13 10 nuclear magnetic resonance spectrum (¹³C-NMR) in solid state and the IR spectrum, recorded on KBr tablet, respectively.

### Example 4

### Preparation of Azithromycin Citrate

20 g of azithromycin dihydrate and 3.5 g of citric acid monohydrate are dissolved at ambient temperature in 50 ml of ethanol, filtered and the solvent is evaporated at low pressure. 24.9 g of a white solid is obtained, containing up to 2.0% of ethanol and up to 7% of water. The X-ray diffraction spectrum confirms that it is an amorphous product (FIG. 10). FIGS. 10, 11 and 12 show the X-ray diffraction spectrum, the carbon 13 nuclear magnetic resonance spectrum (¹³C-NMR) in solid state and the IR spectrum, recorded on KBr tablet, respectively.

### Example 5

### Preparation of Azithromycin Citrate

20 g of azithromycin dihydrate and 3.5 g of citric acid are added to 50 ml of water. The mixture is stirred at ambient temperature and the insoluble material filtered. The solution is concentrated at low pressure to a KF of around 5%, yielding 23.1 g of azithromycin citrate. FIGS. XIII, XIV and XV show the X-ray diffraction spectrum, the carbon 13 nuclear magnetic resonance spectrum (¹³C-NMR) in solid state and the IR spectrum, recorded on KBr tablet, respectively.

### Example 6

### Preparation of Azithromycin Citrate Solutions

Azithromycin citrate solutions are prepared by adding 20 g of azithromycin, 3.5 g of citric acid and the corresponding amount of water (35 to 94 g of water), stirring at ambient temperature for a time ranging between 30 and 60 minutes, and finally filtering to remove insoluble material. The solution is stable at ambient temperature.

Although specific embodiments of this invention have been described and shown, it is obvious that one skilled in the art could introduce variants and alterations, or replace details by others that are technically equivalent without departing from the protection defined by the attached claims.

1. An addition salt comprising: azithromycin and citric acid, in which the molar ratio between the azithromycin and the citric acid is such as to provide a pH between 4.0 and 6.0 in a 10% aqueous solution, and wherein said addition salt is azithromycin hydrogen citrate.

2-3. (canceled)

4. Addition salt of azithromycin according to claim 1 further comprising up to 8 % by weight of water.

5. Addition salt of azithromycin according to claim 1, further comprising up to 6 % by weight of water.

6. Addition salt of azithromycin according to claim 1, which further comprises up to 3 % of residual matter.

7-8. (canceled)

9. Addition salt of azithromycin according to claim 1, wherein when the molar ratio of azithromycin and citric acid is 1:1 a pH of 5 is provided in a 10% aqueous solution.

10-11. (canceled)

12. Process for preparing an addition salt of azithromycin according to claim 1, comprising the steps of:
a) dissolving azithromycin in a solvent or mixture of solvents;
b) adding citric acid; and
c) isolating the product obtained.

13. Process according to claim 12, further comprising that azithromycin is dissolved in monohydrated form in step (a).

14. Process according to claim 12, further comprising that azithromycin is dissolved in dihydrated form in step (a).

15. Process according to claim 12, further comprising that the solvent is selected from: water; the linear or branched C1-C6 aliphatic alcohols, such as methanol, ethanol, n-propanol, isopropanol, n-butanol; cyclic aliphatic alcohols, such as cyclohexanol; diols/such as ethylene glycol, 1,2-propylene glycol, 1,3-propanodiol, 1,4-butanodiol; linear or branched C1-C6 aliphatic ketones, such as acetone, methyl ethyl ketone, methyl isobutyl ketone; cyclic aliphatic ketones, such as cyclohexanone; short-chain aliphatic esters, such as ethyl acetate; short-chain aliphatic ethers, such as ethylic ether, isopropylic ether, etc.; cyclic aliphatic ethers, such as tetrahydrofuran and dioxane, or mixtures thereof.

16. Process according to claim 12, wherein the azithromycin monohydrate or dihydrate is dissolved; and the solvent is selected from water, alcohols, ketones, esters or ethers, or mixtures thereof, preferably water, ethanol, acetone, methyl acetate or tetrahydrofuran, or mixtures thereof.

17. Process according to claim 12, further comprising the step of adding an amount of citric acid is added in step (b) such that the molar ratio between the azithromycin and the citric acid is close to the stoichiometric.

18. Process according to claim 12, for the preparation of azithromycin hydrogen citrate, further comprising in step (c) the salt is isolated by crystallization.

19. Process according to claim 18, wherein step c) further comprises:
c-i) crystallising at a crystallization temperature between 25° C. and the solvent's reflux temperature; and
c-ii) cooling the mixture at a temperature between 0° C. and 25° C., before separating the cystals.

20-21. (canceled)

22. Process for preparing solutions of an addition salt of azithromycin according to claim 1, in water or water-alcohol mixtures containing up to 65% of said salt, which comprises: dissolving the azithromycin hydrogen citrate in water or water-alcohol mixtures and filtering the solution obtained.

23-25. (canceled)

26. A method for the therapeutic treatment of an infection caused by bacteria or protozoans, comprising administering to a mammal in need thereof an effective amount of addition salt of azithromycin according to claim 1.

27. A method for the preventive treatment of an infection caused by bacteria or protozoans, comprising administering to a mammal in need thereof an effective amount of addition salt of azithromycin according to claim 1.

28. Addition salt of azithromycin according to claim 1, wherein said addition salt is in solid state.

29. Addition salt of azithromycin according to claim 2, wherein said addition salt is in crystalline solid state.
